(19) United States
(12) Patent Application Publication
(10) Pub. No.: US 2026/0263632 A1
(43) Pub. Date: Sep. 10, 2026

(54) PHARMACEUTICAL COMPOSITION FOR TREATING AND/OR PREVENTING JOINT DISEASE

(71) Applicant: The University of Osaka, Osaka (JP)

(72) Inventors: Keiji ITAKA, Osaka (JP); Yuta FUKUSHIMA, Tokyo (JP); Jia DENG, Tokyo (JP); Yuki TERAI, Tokyo (JP)

(21) Appl. No.: 18/995,652

(22) PCT Filed: Jul. 19, 2023
(86) PCT No.: PCT/JP2023/026336

§ 371 Date: Jan. 29, 2025

(30) Foreign Application Priority Data
Jul. 30, 2022 (JP) 2022-122392
Aug. 19, 2022 (JP) 2022-131285

(51) Int. Cl. A61K 48/00; A61K 38/17; A61P 19/02

(52) U.S. Cl. CPC A61K 48/0041; A61K 38/1709; A61K 38/1793; A61K 48/005; A61P 19/02

(57) ABSTRACT

The present invention provides a pharmaceutical composition that alleviates pain due to joint disease and suppresses cartilage degeneration. One aspect of the present invention relates to a pharmaceutical composition for treating and/or preventing joint disease that is a polyion complex containing (a) a cationic polymer which is a polymer containing a cationic unnatural amino acid as a monomer unit, in which the cationic unnatural amino acid is an amino acid having a group represented by —(NH—(CH₂)₂)p-NH₂ (where, p is 2, 3, or 4) as a side chain and (b) mRNA that encodes IL-1Ra.

### TECHNICAL FIELD

The present invention relates to pharmaceutical compositions, kits, and such for use in the treatment and/or prevention of joint diseases.

### BACKGROUND ART

Osteoarthritis (OA: osteoarthritis), which is known as joint disease, is a chronic degenerative disease of the joints that progresses when the cartilage tissue lacking autonomous regenerative capacity is exposed to inflammation or mechanical stress, resulting in gradual degeneration. Osteoarthritis is a major health problem especially among the elderly, and the pain and discomfort it causes can reduce a patient's quality of life. However, disease-modifying osteoarthritis drugs (DMOADs: disease-modifying osteoarthritis drugs) have not yet been developed for clinical use.

Anakinra, which is a recombinant protein formulation of interleukin 1 receptor antagonist (IL-1Ra), is known as a therapeutic drug for rheumatoid arthritis (RA), a joint disease with swelling and pain in the joints (Non-Patent Document 1, 2). In Non-Patent Document 1, it is described that the half-life of intra-articularly injected anakinra was less than 4 hours based on pharmacokinetic analysis.

Drug delivery systems that deliver drugs to suitable locations in the body are being researched and developed as provision of new medicines with fewer side effects. Among them, drug delivery systems using polyion complexes (hereinafter also referred to as “PICs”) have attracted attention as a technology that can encapsulate drugs in nanomicelles and deliver drugs in a disease site-specific manner. Patent Document 1 describes that a polyion complex consisting of an mRNA encoding the transcription factor Runx1 and a cationic polymer was able to suppress cartilage degeneration caused by osteoarthritis.

### CITATION LIST

### Patent Documents

Patent Document 1: WO2017/022665

### Non-Patent Documents

Non-Patent Document 1: Chevalier, X., et al. (2009). Intraarticular injection of anakinra in osteoarthritis of the knee: a multicenter, randomized, double-blind, placebo-controlled study. Arthritis Rheum 61, 344-352. 10.1002/art.24096.
Non-Patent Document 2: Martel-Pelletier, J., Pelletier, JP. Nat Rev Rheumatol5, 363-364 (2009). A single injection of anakinra for treating knee OA?

### SUMMARY OF THE INVENTION

### Problems to be Solved by the Invention

However, the pharmaceutical compositions described in Patent Document 1 were not sufficient to alleviate pain, although they were effective in suppressing cartilage degeneration caused by osteoarthritis. In addition, the anakinra described in Non-Patent Documents 1 and 2 is problematic in that the protein has a short half-life of less than 4 hours in tissues with a single administration, and its pain-relieving effect is also short-lived.

The objective of the present invention is to provide pharmaceutical compositions for treating and/or preventing joint diseases, which suppress cartilage degeneration and are effective for a long period of time in pain reduction.

### Means for Solving the Problems

The preferred aspects of the present embodiment are as follows.

1. A pharmaceutical composition for use in the treatment and/or prevention of joint diseases, which is a polyion complex comprising: (a) a cationic polymer, which is a polymer comprising a cationic unnatural amino acid as a monomer unit, wherein the cationic unnatural amino acid is an amino acid having a group represented by —NH—(CH₂)₂)ₚ—NH₂ (wherein p is 2, 3 or 4) as a side chain, and (b) an mRNA encoding IL-1Ra.
2. The pharmaceutical composition described according to 1 above, further comprising an mRNA encoding Runx1.
3. The pharmaceutical composition described according to 1 or 2 above, wherein the cationic polymer is a block copolymer comprising a block based on polyethylene glycol and a block based on a polymer comprising a cationic unnatural amino acid as a monomer unit.
4. The pharmaceutical composition described according to any of 1 to 3 above, in which the cationic polymer is represented by formula (I) below:

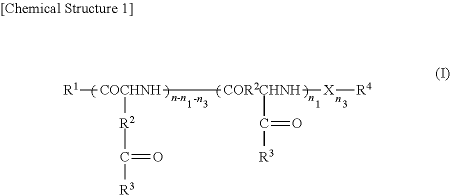

wherein in formula (I),
R¹ is a group based on polyethylene glycol, and the polyethylene glycol and the adjacent amino acid may be bonded via a linker;
R² is a methylene group or an ethylene group;
R³ is a group represented by —NH—(CH₂)₂)ₚ—NH₂, and p is 2, 3 or 4;
R⁴ is hydrogen, a protecting group, a hydrophobic group or a polymerizable group;
X is a group based on a cationic amino acid;
n is any integer of 2 to 5000;
n₁ is any integer of 0 to 5000;
n₃ is any integer of 0 to 5000;
and n-n₁-n₃ is any integer greater than or equal to 0; and wherein each repeating unit in the formula is shown in a specific order for convenience of description, but each repeating unit can exist in any order, and each repeating unit may exist randomly, and each repeating unit may be the same or different.
   5. The pharmaceutical composition described according to any one of 1 to 4 above, wherein the joint disease is arthritis, osteoarthritis, or rheumatoid arthritis.
   6. A method for treating and/or preventing joint diseases, comprising the step of administering to a subject a pharmaceutical composition described according to any of 1 to 5 above
   7. A method for manufacturing a pharmaceutical composition for the treatment or prevention of joint diseases, comprising the step of forming a polyion complex that comprises a cationic polymer and an mRNA encoding IL-1Ra, wherein the cationic polymer is a polymer comprising a cationic unnatural amino acid as a monomer unit, and the cationic unnatural amino acid is an amino acid having a group represented by —NH—(CH₂)₂)ₚ—NH₂ (wherein p is 2, 3, or 4) as a side chain.

### Effects of the Invention

According to one aspect of the present embodiment, pharmaceutical compositions for treating and/or preventing joint diseases that are effective for a long period of time in inhibiting cartilage degeneration and alleviating pain.

### BRIEF DESCRIPTION OF THE DRAWINGS

FIG. 1 is a diagram showing the protein expression from an mRNA encoding Zsgreen1 administered to the temporomandibular joint, as observed by immunofluorescence staining.

FIG. 2A is a graph showing the results of measuring changes in the expression level of an mRNA encoding IL-1Ra administered to the temporomandibular joint by qRT-PCR.

FIG. 2B is a diagram showing the protein expression from an mRNA encoding IL-1Ra administered to the temporomandibular joint, as confirmed by Western blotting.

FIG. 3A is a diagram illustrating a process for examining the effects of administering an mRNA encoding IL-1Ra to rats in which temporomandibular joint osteoarthritis (TMJOA) had been induced.

FIG. 3B is a graph showing the results of evaluating the effects of administering an mRNA encoding IL-1Ra to rats in which TMJOA has been induced, by using the rat's head withdrawal threshold (HWT).

FIG. 3C is a diagram showing the effects of administering an mRNA encoding IL-1Ra to rats in which TMJOA has been induced, as observed by micro-CT of the subchondral bone of the temporomandibular joint.

FIG. 4A is a diagram showing the effects of administering an mRNA encoding IL-1Ra to rats in which TMJOA has been induced, as observed by HE, TB, and SO staining of the cartilage of the temporomandibular joint.

FIG. 4B is a graph showing the effects of administering an mRNA encoding IL-1Ra to rats in which TMJOA has been induced, by using Mankin score.

FIG. 4C is a schematic diagram showing the measurement area for measuring cartilage degeneration in the condyle of a rat temporomandibular joint.

FIG. 4D is a graph showing the results of measuring the cartilage thickness for each region of the temporomandibular joint of a rat in which TMJOA has been induced. The results at 2 weeks and 4 weeks following mRNA administration are shown.

FIG. 5 is a graph showing the results of measuring the gene expression levels of IL-6 and TNF-α following the administration of an mRNA encoding IL-1Ra to rats in which TMJOA has been induced

FIG. 6 is a graph showing pain evaluation following the administration of a PIC micelle solution containing each mRNA to rats in which osteoarthritis (OA) of the knee joint has been induced.

FIG. 7 is a diagram showing the knee joint of a rat in which OA of the knee joint has been induced, as observed by Fast Green Safranin O staining.

FIG. 8 shows the sequence of a cDNA encoding IL-1Ra used in the Examples.

FIG. 9 shows the sequence of a cDNA encoding Runx1 used in the Examples.

FIG. 10 is a graph showing pain evaluation following the administration of a PIC micelle solution containing each mRNA to rats in which osteoarthritis (OA) of the knee joint has been induced.

FIG. 11 shows the results of measuring the anteroposterior diameter and left and right diameter of the knee joint at the center of the patellar ligament, and plotting the mean value as the diameter in order to evaluate the degree of swelling of the OA knee (knee diameter).

FIG. 12 is a diagram showing the knee joint of a rat in which OA of the knee joint has been induced, as observed by Fast Green Safranin O staining.

### MODE FOR CARRYING OUT THE INVENTION

### <Pharmaceutical Composition>

One aspect of the present embodiment relates to a polyion complex (also described as “PIC”) comprising: (a) a cationic polymer and (b) an mRNA encoding an interleukin-1 receptor antagonist (also described as “IL-1Ra”) for use in the treatment and/or prevention of joint diseases. As used in the present specification, mRNA refers to messenger RNA. The pharmaceutical composition of the present embodiment preferably forms a polyion complex in which the polycation moiety of a cationic polymer is bound to an mRNA by electrostatic interactions, preferably in a solution (more preferably in an aqueous solution).

As described below, the cationic polymer may be a block copolymer of a block based on polyethylene glycol (PEG) and a block based on cationic polyamino acids. In a preferred embodiment, the structure is such that the mRNA and the polycation moiety of the block copolymer are condensed by electrostatic interactions to form a core portion, and the hydrophilic and biocompatible PEG portion in the block copolymer forms a shell portion around the core portion. In the present specification, particles having such a core-shell structure are also referred to as “polyion complex (PIC) micelles”. Since PIC micelles have a particle size of several tens of nanometers, they have high permeability into tissues. In addition, PIC micelles stably encapsulate mRNA with the core-shell structure and can avoid foreign body recognition mechanisms in living organisms.

### <(i) Cationic Polymer>

In the present embodiment, the cationic polymer is a polymer comprising an unnatural amino acid as a monomer unit, and the unnatural amino acid has a group represented by —NH—(CH₂)₂)ₚ—NH₂ as a side chain {where p is 2, 3 or 4}. p is preferably 2 or 3.

In a preferred embodiment, the cationic polymer is a block copolymer having a block based on polyethylene glycol (PEG) and a block based on cationic polyamino acids, and is also referred to simply as a “block copolymer” in the present specification.

The cationic polymer may comprise, in addition to the predetermined unnatural amino acids mentioned above, cationic natural amino acids and/or other cationic unnatural amino acids as monomer units, and it is preferred that natural amino acids and/or unnatural amino acids having a cationic group in their side chains are included as monomer units. Here, the cationic group is not limited to a group that is already cationic by hydrogen ion coordination, but also includes groups that become cationic if a hydrogen ion is coordinated to them. The cationic natural amino acids are, for example, preferably histidine, tryptophan, ornithine, arginine and lysine, more preferably arginine, ornithine and lysine, further preferably ornithine and lysine, and even more preferably lysine.

In a cationic polymer, the polymeric moiety having an unnatural amino acid as a monomer unit can be obtained, for example, by introducing a group represented by —NH—(CH₂)₂)ₚ—NH₂ {where p is 2, 3, or 4} into a polymer containing aspartic acid or glutamic acid as a monomer unit. A person skilled in the art can obtain the polymeric moiety having this unnatural amino acid as a monomer unit, by reacting a polymer containing aspartic acid or glutamic acid as a monomer unit with diethylenetriamine, triethylenetetraamine or tetraethylenepentamine.

The cationic polymer includes, but is not limited to, for example, the general formula (I) below; and preferably it is a compound represented by:

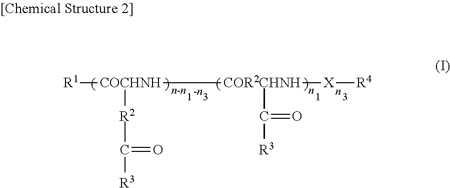

wherein in formula (I),
R¹ is a group based on polyethylene glycol, and the polyethylene glycol and the adjacent amino acid may be bonded via a linker;
R² is a methylene group or an ethylene group;
R³ is a group represented by —NH—(CH₂)₂)ₚ—NH₂, and p is 2, 3 or 4;
R⁴ is hydrogen, a protecting group, a hydrophobic group, or a polymerizable group;
X is a group based on a cationic amino acid;
n is any integer of 2 to 5000;
n₁ is any integer of 0 to 5000;
n₃ is any integer of 0 to 5000; and n-n₁-n₃ is any integer greater than or equal to 0, and wherein each repeating unit in the formula is shown in a specific order for convenience of description, but each repeating unit can exist in any order, and each repeating unit may exist randomly, and each repeating unit may be the same or different.

In the formula (I) above, p is 2, 3 or 4, and preferably 2 or 3 in one of the embodiments.

In the formula (I) above, examples of the protecting group include a C₁₋₆ alkylcarbonyl group or such, preferably an acetyl group; examples of the hydrophobic group include groups based on benzene, naphthalene, anthracene, pyrene, and derivatives thereof and a C₁₋₆ alkyl group or such; and examples of the polymerizable group include a methacryloyl group, an acryloyl group and such. Methods for introducing these protecting groups, hydrophobic groups and polymerizable groups into block copolymers are well known to those skilled in the art.

In the formula (I) above, the polyethylene glycol (PEG) that constitutes R¹ preferably has an average degree of polymerization of 5 to 20,000, more preferably 10 to 5,000, and still more preferably 40 to 500, and there is no particular limitation as long as the polyion complex formation between the cationic polymer and mRNA is not inhibited. The terminus of the PEG structural portion of the cationic polymer may be protected with a hydroxyl group, a methoxy group, or a protecting group.

In the formula (I) above, the linker is, for example, —(CH₂)ᵣ—NH— {where r is any integer of 1 to 5} or —(CH₂)ₛ—CO— {where s is any integer of 1 to 5}, and preferably can be bound to adjacent amino acids of formula (I) via a peptide bond. Further, the linker may preferably be bound to PEG on the methylene side of the PEG via the O atom of the PEG.

In the formula (I) above, n is any integer of 2 to 5000, preferably any integer of 2 to 3000, and more preferably any integer of 2 to 500. n₁ is any integer of 0 to 5,000, for example, preferably any integer of 0 to 3,000, and more preferably any integer of 0 to 500. n₃ is any integer of 0 to 5,000, for example, preferably any integer of 0 to 3,000, and more preferably any integer of 0 to 500. Further, n-n₁-n₃ is any integer greater than or equal to 0.

In one embodiment, the cationic polymer is preferably a block copolymer of PEG-linker-polycation block (where the PEG, linker and polycation block are as defined above).

When the cationic polymer used to form the polyion complex is a polymer comprising a monomer unit having a group represented by —(NH—(CH₂)₂)ₚ—NH₂ {where p is 2, 3, or 4} as a side chain, the mRNA is more likely to be present in the body for a long period of time. In one embodiment, when p is 3 or 4, the mRNA may be highly expressed in the body for a significantly long period of time. In the Examples of the present invention, the cationic polymer comprises a monomer unit having a group represented by —(NH—(CH₂)₂)ₚ—NH₂ {where p is 2, 3 or 4} as a side chain; and in one embodiment, it can be a cationic polymer where p is more preferably 2 or 3.

(ii) mRNA

The mRNA constituting the pharmaceutical composition of the present embodiment comprises an mRNA encoding IL-1Ra (also referred to as “an mRNA of IL-1Ra” or “an IL-1Ra mRNA”). IL-1Ra is thought to suppress IL-1 activity by competitively inhibiting the binding of IL-1 to the IL-1 receptor. IL-1Ra is found in various animals (mammals) such as humans, mice, and rats. By using known databases that store information on proteins or information on genes, it is possible to obtain information on the amino acid sequence, nucleotide sequence encoding the amino acid sequence, isoforms, and polymorphisms for IL-1Ra derived from various animals. In addition to the open reading frame, the mRNA may comprise a 5′ cap, 5′ UTR, 3′ UTR, poly(A) tail and such.

The inventors of the present invention have found that administering a PIC micelle solution that encapsulates an mRNA of IL-1Ra to a subject with induced arthritis is remarkably effective for suppressing cartilage degeneration and alleviating pain. With a single administration of the pharmaceutical composition of the present embodiment, the protein expression is long-lasting, and the pain reduction effect also lasts for a long period of time. In the example described below, with a single administration, the expression of the IL-1Ra protein was confirmed for 36 hours and sustained pain reduction was demonstrated for 4 weeks. This is a very superior effect compared to the half-life of anakinra, a known recombinant protein preparation, which is 4 hours. Although the detailed mechanism of action is unknown, it is possible that the extracellularly secreted IL-1Ra protein and the intracellularly present IL-1Ra protein cooperatively regulate inflammatory signals.

In one aspect of the present embodiment, the mRNA may include other mRNAs besides the IL-1Ra mRNA. As the other mRNA, mRNAs encoding proteins that are effective in reducing inflammation and/or pain in joint diseases are preferred.

In one aspect of the present embodiment, the pharmaceutical composition preferably comprises an mRNA encoding Runx1 (Runt-related Transcription Factor 1) (“Runx1 mRNA”), in addition to the IL-1Ra mRNA. Runx1 is a transcription factor known to control cartilage formation in embryos and adults. By including an IL-1Ra mRNA and Runx1 mRNA, the effect of suppressing cartilage degeneration can be further enhanced in addition to the effect of alleviating pain in joint diseases.

In the present embodiment, cytidine and uridine in the mRNA may be modified. The modified cytidine includes, for example, 5-methyl-cytidine, and the modified uridine includes pseudouridine, 2-thio-cytidine and such. In one aspect, the modified cytidine and modified uridine may be included in an amount of more than 10 mol %, 20 mol %, or 30 mol % of the total cytidine and uridine. Further, the mRNA may be a full-length sequence, a partial sequence, or a mutated sequence encoding the protein of interest (IL-1Ra, Runx1, or such). It may also be a sequence in which the coding amino acids are not changed, but the corresponding codons are modified. The mRNA may also comprise a 5′ cap, 5′ UTR, 3′ UTR, poly(A) tail or such in addition to the open reading frame.

The mixing ratio of the cationic polymer (preferably a block copolymer) to mRNA is not limited, and for example, the ratio of the total number (N) of cationic groups (amino groups) in the cationic polymer (preferably a block copolymer) to the total number (P) of phosphate groups in the mRNA (N/P ratio) is preferably 1.5 to 60, more preferably 1.5 to 32, and even more preferably 2 to 10. In this case, N is the total number of primary amines and secondary amines in the side chain of the polycationic moiety. When the N/P ratio is within the above range, it is preferable because there is no presence of free polymer and high expression efficiency can be obtained in vivo. Further, the cationic group (N) described above refers to a group that can form ionic bonds through electrostatic interactions with a phosphate group in the encapsulated mRNA. When multiple types of mRNA are included, the total number of phosphate groups (P) described above refers to the total number of phosphate groups in all the mRNAs.

The pharmaceutical compositions of the present embodiment are preferably used in the state of formed PIC micelles in solution, as described above. The size of the PIC micelles is not limited, but the particle size by dynamic light scattering (DLS) measurement is, for example, preferably 20 to 150 nm and more preferably 30 to 100 nm.

### <Target Diseases>

The pharmaceutical compositions of the present embodiment may be used effectively in the treatment and prevention of a joint disease. Examples of a joint disease include arthritis, osteoarthritis (OA), and rheumatoid arthritis (RA). In one aspect, the pharmaceutical compositions of the present embodiment may be suitably used in the treatment and/or prevention of osteoarthritis.

One aspect of the present embodiment relates to the use of the above pharmaceutical composition in the manufacture of a pharmaceutical for the treatment and/or prevention of joint diseases. Also, an aspect of the present embodiment relates to a method of treating and/or preventing a joint disease, comprising the step of administering a therapeutically effective amount of the above pharmaceutical composition to a subject in need of treatment.

The pharmaceutical composition of the present embodiment may contain other ingredients in addition to the polyion complex described above. The other ingredients in the pharmaceutical compositions of the present invention are not particularly limited and may be selected according to the purpose, for example, a pharmaceutically acceptable carrier or additive. There are no particular limitations on the carriers or additives, which may include, for example, diluents, excipients, suspending agents, lubricants, vehicles, emulsifiers, tablet degrading substances, absorbents, preservatives, preservatives, surfactants, coloring agents, or fragrances. The amount of other ingredients in the pharmaceutical compositions of the present invention is also not limited and can be selected according to the purpose.

The dosage form of the pharmaceutical compositions of the present invention is not particularly limited and can be suitably selected according to the desired method of administration, for example, injectable formulations (solution, suspension, solid formulation for dissolution for use, and such). For injections, for example, a pH adjusting agent, a buffering agent, a stabilizing agent, an isotonic agent, a local anesthetic, or such is added to the composition, and injections for subcutaneous, intraarticular, intramuscular, intravenous use or such can be manufactured using conventional methods. Examples of the pH adjusting agent and the buffering agent include sodium citrate, sodium acetate, sodium phosphate, HEPES buffer and such. The stabilizing agents include, for example, sodium pyrosulfite, EDTA, thioglycolic acid, thiolactic acid, and such. The isotonic agents include, for example, sodium chloride, glucose and such. The local anesthetics include, for example, procaine hydrochloride, lidocaine hydrochloride and such. The solid dosage form may be provided with an enteric coating.

The method of administering the pharmaceutical composition according to the present invention is not particularly limited, and for example, either local administration or systemic administration can be selected depending on the dosage form of the pharmaceutical composition, the condition of the patient, and such. Administration can be carried out, for example, by intraarticular administration, intravenous administration, transarterial administration, subcutaneous administration, intramuscular administration, oral administration, pulmonary administration, enteral administration, enema administration, tube feeding, and such.

The subject of administration of the pharmaceutical composition according to the present invention is not particularly limited and can be selected according to the purpose. For example, the subject may be a human or a non-human mammal such as a mouse, rat, cow, horse, pig, monkey, dog, cat, or such, but it is preferably a human, in particular, a human patient suffering from a joint disease. The subject may be a healthy subject or a subject suffering from some type of disease. Further, the pharmaceutical compositions may be administered for the purpose of preventing the onset of joint disease, and in particular for the purpose of preventing recurrence.

The dosage of the pharmaceutical composition according to the present invention is not particularly limited and can be suitably selected according to the dosage form, the age and weight of the subject of administration, the degree of desired effect, and such. The dosage of the pharmaceutical composition according to the present invention, for example, can be set so that the amount of mRNA per dose is 1 μg to 1 mg.

There is no particular limitation as to the timing of administration of the pharmaceutical compositions of the present invention, which may be selected as appropriate for the purpose, for example, it may be administered prophylactically to patients susceptible to the above diseases, or it may be administered therapeutically to patients presenting symptoms. The number of times of administration is also not particularly limited and may be selected as appropriate depending on the subject of administration, the age and weight of the subject, the degree of desired effect, or such.

As shown in the Examples below, when the pharmaceutical composition of the present embodiment is administered to a subject, the expression period of IL-1Ra protein is long, so that the number of administrations can be reduced compared to the recombinant protein preparation Anakinra, and the patient's burden of treatment will be significantly reduced. The frequency of administration can be appropriately determined according to the characteristics of the subject to be administered, the dosage per dose, and such. In one aspect of the present embodiment, for example, the administration interval is preferably 1 day to 2 months, more preferably 3 days to 1 month, but it is not limited thereto.

One aspect of the present embodiment is a pharmaceutical composition for the treatment or prevention of arthritis (for example, osteoarthritis or rheumatoid arthritis), comprising a polyion complex micelle comprising:
(i) a cationic polymer wherein in formula (I), R¹ is a group based on protected polyethylene glycol, p is 2 or 3, and R⁴ is hydrogen or a protected group; and
(ii) an IL-1Ra mRNA.

One aspect of the present embodiment is a pharmaceutical composition for the treatment or prevention of arthritis (for example, osteoarthritis or rheumatoid arthritis), comprising a polyion complex micelle comprising:
(i) a cationic polymer wherein in formula (I), R¹ is a group based on protected polyethylene glycol, p is 2 or 3, and R⁴ is hydrogen or a protected group; and
(ii) an IL-1Ra mRNA and an Runx1 mRNA.

In one aspect of the present embodiment, a kit for preparing a pharmaceutical composition for use in the treatment and/or prevention of joint diseases is provided. The kit of the present embodiment may include the cationic polymer and the IL-1Ra mRNA described above, which may be stored (preserved) in separate containers. For the cationic polymer and the IL-1Ra mRNA, the description of the pharmaceutical composition described above applies. The kit may also include an mRNA of Runx1 in addition to the IL-1Ra mRNA.

In the kit, the storage state of the cationic polymer is not limited and can be selected from a solution state or powder state, or such, taking its stability (preservability) and ease of use into consideration. The cationic polymer and IL-1Ra mRNA may be mixed or stored separately. If the cationic polymer and IL-1Ra mRNA are stored separately, instructions describing the conditions for mixing them may be included. The kit may also include various buffers (for example, buffer for dissolving the cationic polymer and/or mRNA).

### EXAMPLES

Hereinafter, the present embodiment will be described in detail with reference to the examples, but the present embodiment is not limited to the examples below.

The abbreviations used in the examples are as follows.
TMJ: temporomandibular joint
OA: osteoarthritis
TMJOA: temporomandibular joint osteoarthritis
MIA: monoiodoacetic acid
IL-1Ra mRNA: an mRNA encoding IL-1Ra
Luc2 mRNA: an mRNA encoding luciferase 2
Runx1 mRNA: an mRNA encoding Runx1

### Example 1: Preparation of Polyion Complex (PIC)

A polyion complex comprising a cationic polymer and an mRNA was prepared.

### Example 1-1: Synthesis of the PEG-pAsp (DET) Block Copolymer

The polymer (pAsp (DET)) was obtained by introducing a group represented by —(NH—(CH₂)₂)₂—NH₂ into the side chain of polyaspartic acid. Specifically, polyethylene glycol (MeO-PEG-NH₂) (Nippon Yushi) with an average molecular weight of 12,000, which has a methoxy group at one end and an aminopropyl group at the other end, was dissolved in methylene chloride. β-benzyl-L-aspartate-N-carboxylic anhydride (BLA-NCA) (Chuo Kasei Co., Ltd.) was added to and dissolved in a mixture of N,N-dimethylformamide (DMF) and the above methylene chloride solution to obtain a reaction solution. Next, the reaction solution was then reacted at 40° C. for 2 days to obtain a polyethylene glycol-poly(β-benzyl-L-aspartate) block copolymer (MeO-PEG-PBLA). The degree of polymerization of the PBLA moiety was 63 based on analysis by ¹H-NMR (JEOL EX300 spectrometer, JEOL (JAPAN ELECTRON OPTICS LABORATORY CO., LTD)).

Next, MeO-PEG-PBLA was reacted with diethylenetriamine to obtain a MeO-PEG-pAsp (DET) block copolymer. Specifically, MeO-PEG-PBLA was dissolved in benzene and lyophilized. The lyophilized MeO-PEG-PBLA was dissolved in N,N-dimethylformamide (DMF). After that, diethylenetriamine (purchased from Wako Pure Chemicals) was added dropwise to each of the resulting solutions and the reaction was carried out under mild anhydrous conditions at 40° C. to obtain a MeO-PEG-pAsp (DET) block copolymer (simply referred to as “PEG-PAsp (DET) block copolymer”). The resulting block copolymer showed a sharp single molecular weight distribution (Mw/Mn=1.04) by gel permeation chromatography (GPC).

### Example 1-2: mRNA Preparation

(Preparation of an IL-1Ra mRNA)

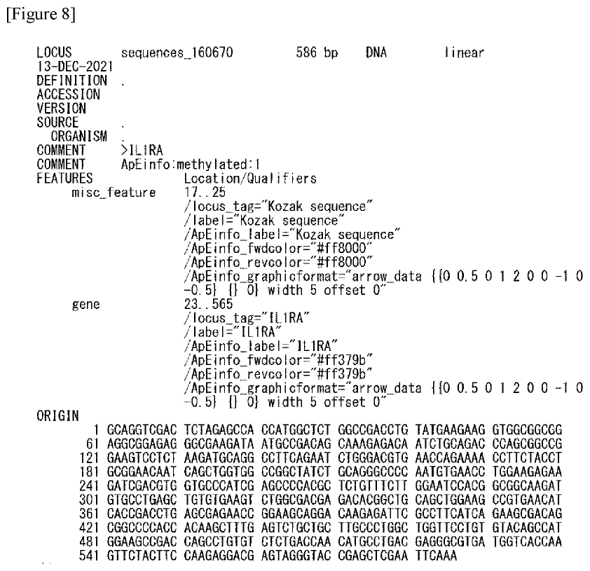

First, a human IL-1Ra expression vector was prepared. A cDNA encoding untagged human IL-1Ra (SEQ ID NO.: 1, FIG. 8, GenBank accession number: NM_173842) was purchased from Thermo Fisher Scientific. To create a DNA template for in vitro transcription, the coding region of human IL-1Ra was cloned into the pSP73 vector (manufactured by Promega Corporation) for expression under the T7 promoter. A 120 bp polyA/T sequence was cloned downstream of the protein coding sequence of the vector to bind the polyA chain to the 3′ terminus of the mRNA. In vitro transcription was performed with linearized pSP73-IL-1Ra-Poly (A) using the mMESSAGE mMACHINE T7 Ultra Kit (Ambion). Unmodified natural-type nucleobases were used for the mRNA production. The quantity and quality of transcribed mRNA were measured using a Nanodrop 2000 spectrophotometer (Thermo Fisher Scientific) and an Agilent 2100 Bioanalyzer chip-based capillary electrophoresis system (Agilent Technologies), respectively.

(Preparation of a Runx1 mRNA)

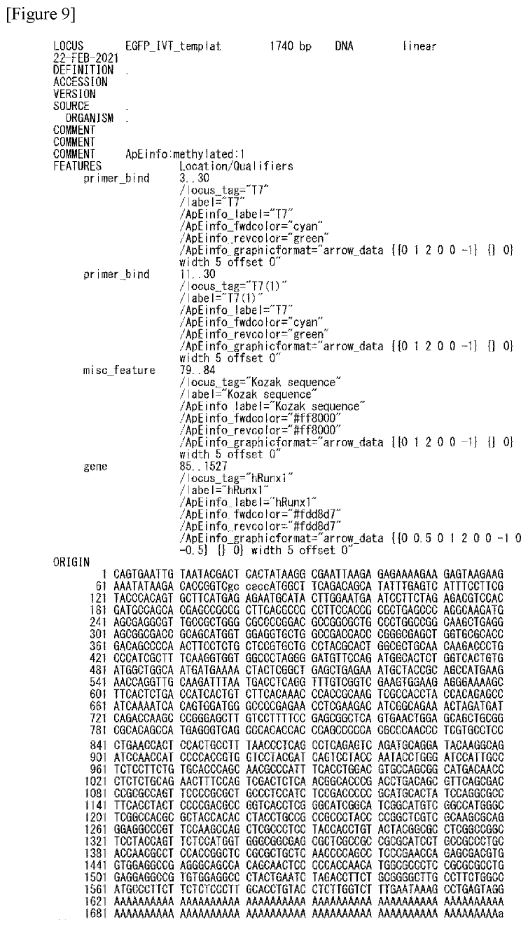

A cDNA encoding human Runx1 (SEQ ID NO.: 10, FIG. 9, GenBank accession number: NM_001754.4) was cloned into the pSP73 vector (manufactured by Promega Corporation) for expression under the T7 promoter, and a Runx1 mRNA was prepared by the same method as for the IL-1Ra mRNA described above.

(Preparation of a Luc2 mRNA and Zsgreen1 mRNA)

A Luc2 mRNA was prepared using the protein transcription region of luciferase 2 (Luc2) (pGL4.13, manufactured by Promega Corporation) by the same method as for the IL-1Ra mRNA described above. Further, the protein transcription region of green fluorescent protein (Zsgreen1) (pZsGreen1-N1, manufactured by Takara Bio Inc.) was used to prepare a Zsgreen1 mRNA by the same method as for the IL-1Ra mRNA described above.

### Example 1-3: Preparation of a Polyion Complex

A solution of polyion complex micelles (PIC micelle solution) was obtained by mixing the PEG-pAsp (DET) block copolymer obtained in (Example 1-1) as described above with the IL-1Ra mRNA obtained in (Example 1-2) as described above in 10 mM HEPES buffer (pH 7.3). The concentration of mRNA was 75 μg/mL, and the mixing ratio (N/P) of the amino group (N) of the cationic group in the block copolymer to the phosphate group (P) in the nucleic acid was 8. The final mRNA concentration in the PIC micelle solution was 50 μg/mL.

A PIC micelle solution containing the Runx1 mRNA, a PIC micelle solution containing the Luc2 mRNA, and a PIC micelle solution containing the Zsgreen1 mRNA were prepared in the same way.

### Example 2: Administration of an IL-1Ra mRNA to a Temporomandibular Joint Disease Model Animal (Rat)

### Example 2-1: Model Animal Preparation and mRNA Administration

In Example 2, a rat model of temporomandibular joint osteoarthritis (TMJOA) was used as a model animal for osteoarthritis induced by intra-articular administration of monoiodoacetic acid (MIA). Male SD rats (8 weeks old, average weight 240 g, Sankyo Labs) were used as the model animal. Rats were housed at room temperature on a 12-hour light/12-hour dark cycle and had free access to food and water. In the TMJOA model, TMJOA was induced by intra-articular injection of MIA (manufactured by Sigma-Aldrich). Specifically, TMJOA was induced under 2% isoflurane anesthesia, by injecting 0.5 mg of MIA dissolved in 50 μL of PBS into the superior joint cavity of the left and right temporomandibular joints using a 30-gauge needle.

In the TMJOA model, MIA injected into the joint cavity triggers intra-articular inflammation and induces the formation of inflammatory cells, osteoclasts and the like. This leads to degeneration and destruction of the subchondral bone, followed by degeneration of the cartilage leading to progression of osteoarthritis (OA). Continuous pain also occurs in the MIA-induced TMJOA model.

Hereinbelow, when administering an mRNA in (Example 2-2) to (Example 2-6), a solution of PIC micelles encapsulating the mRNA (50 μL of a PIC micelle solution containing 2.5 μg of mRNA) obtained in (Example 1-3) as described above was administered. When the mRNA was administered to the TMJOA model, 50 μL of a PIC micelle solution containing 2.5 μg of mRNA was administered to the temporomandibular joint (TMJ) 24 hours after MIA injection for evaluation.

### Example 2-2: Protein Expression from the Administered mRNA

To investigate the delivery of the administered mRNA and protein expression, a PIC micelle solution encapsulating the Zsgreen1 mRNA was injected into the temporomandibular joint of normal rats, and 24 hours after injection, the distribution of protein expression was visualized by immunofluorescence staining. Comparison was made with those injected with a PIC micelle solution containing the Luc2 mRNA as a negative control. For immunofluorescence staining, sections of the temporomandibular joint were incubated with an antibody against Zsgreen1 (1:250, rabbit polyclonal, Takara Bio) and an Alexa Fluor-488 goat anti-rabbit secondary antibody (1:250, Invitrogen). Subsequently, the sections were then counterstained with DAPI (Thermo Fisher Scientific) and observed under an inverted fluorescence microscope (BZ9000; Keyence Co.).

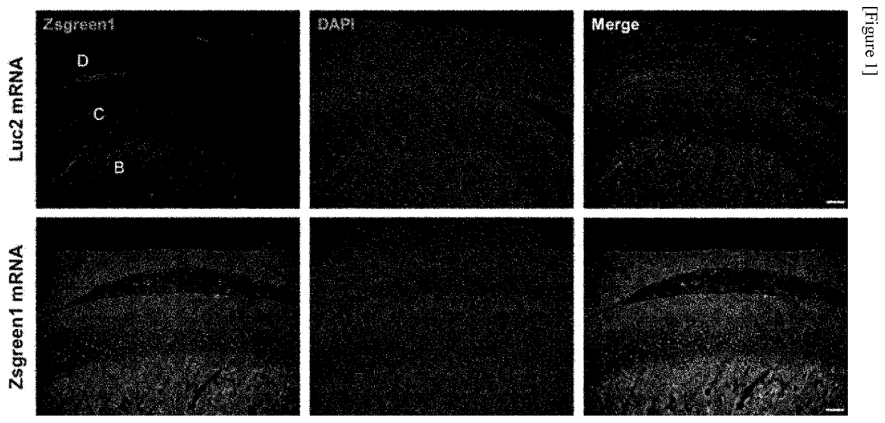

FIG. 1 shows the results of immunofluorescence staining with an anti-Zsgreen1 antibody and DAPI. In FIG. 1, D represents Disc (articular disc), C represents Cartilage (cartilage), B represents Bone (bone), and the scale bar represents 100 μm. The green signal of Zsgreen1 was observed in the articular disc (D) and cartilage (C) (FIG. 1). This revealed that the PIC micelles containing mRNA diffuse widely within the osteochondral tissue to express the protein.

### Example 2-3: Delivery of the IL-1Ra mRNA to Cartilage and IL-1Ra Protein Expression

Delivery of the IL-1Ra mRNA to articular cartilage and IL-1Ra protein expression were evaluated. The IL-1Ra mRNA was administered to rats without MIA administration and to rats in which TMJOA had been induced by MIA administration. The samples at 12 hours, 24 hours, 36 hours, and 48 hours after administration of the IL-1Ra mRNA were subjected to qRT-PCR (primer set is SEQ ID NOs.: 2 and 3) to calculate the relative amount of the IL-1Ra mRNA detected in the cartilage tissue.

The qRT-PCR was performed using the following procedure. First, total RNA was isolated from only the articular discs and cartilage of the condylar heads using the RNeasy Fibrous Tissue Kit (manufactured by Qiagen). In order to extract sufficient RNA, the bilateral condylar heads of each rat were made into one sample. Immediately after dissection, the tissue was frozen in liquid nitrogen and homogenized using a multi-bead shocker (manufactured by Yasui Kikai Co., Ltd.). Reverse transcription was performed using the PrimeScript RT Master Mix (manufactured by Takara Bio). qRT-PCR was performed using the StepOnePlus ((trademark) Real-time PCR System (Applied Biosystems) with the PowerTrack (trademark) SYBR (trademark) Green Master Mix (manufactured by Applied Biosystems). Amplification specificity was confirmed using a melting curve. The relative mRNA expression levels were normalized with the amount of β-actin, a housekeeping gene (primer set is SEQ ID NOs.: 4 and 5), and calculated using the ΔΔCT method. The primer sequences used are listed in Table 1.

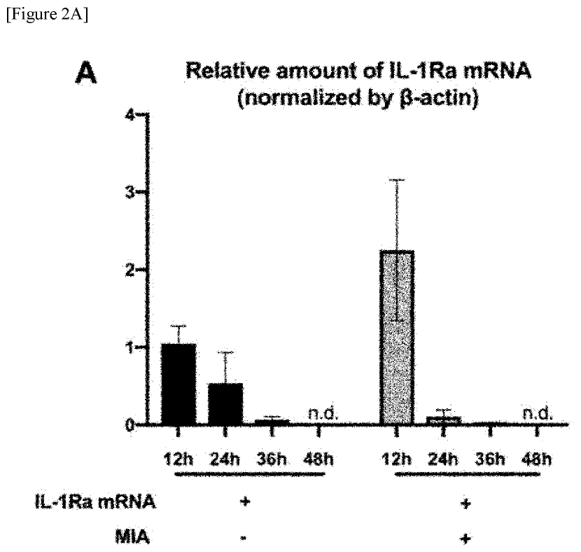

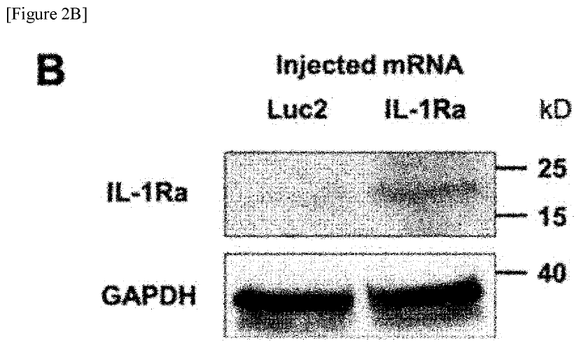

FIG. 2A shows the results of qRT-PCR (N=3/group, data are expressed as mean±SEM (standard error of the mean)). In FIG. 2A, the left side shows the results from normal rats to which MIA was not administered and the right side shows the results from rats to which MIA was administered and inflammation caused by TMJOA. “n.d.” denotes not detected.

As FIG. 2A shows, the relative amount of the IL-1Ra mRNA detected in cartilage was highest 12 hours after mRNA administration. The relative amount of the IL-1Ra mRNA decreased gradually but was still detectable 36 hours after injection. The IL-1Ra mRNA was at an undetectable level by qRT-PCR 48 hours after injection. Further, it was shown that even under inflammatory conditions caused by MIA, the IL-1Ra mRNA was delivered into the cartilage tissue as in the case without MIA administration.

FIG. 2B shows the amount of IL-1Ra protein produced 24 hours after administration of the IL-1Ra mRNA to normal rats, as evaluated by Western blotting. A PIC micelle solution containing the luciferase 2 (Luc2) mRNA was administered as a negative control.

Western blotting was performed according to the procedure below. In order to extract sufficient protein, the bilateral condylar heads of each rat were made into one sample. Total protein was recovered from the articular discs and cartilage of the condylar heads. The tissue was frozen immediately after dissection, and homogenized using a multi-bead shocker (manufactured by Yasui Instruments, Inc.) after addition of a mixture of RIPA buffer and Pierce (trademark) Protease Inhibitor Tablets (manufactured by Thermo Fisher Scientific). The total protein concentration was determined using the Pierce (trademark) BCA protein assay kit (manufactured by Thermo Fisher Scientific). 40 μg of protein sample was subjected to sodium dodecyl sulfate-polyacrylamide gel electrophoresis using the MES SDS running buffer (manufactured by Thermo Fisher Scientific). The blot was then transferred to a polyvinylidene fluoride membrane using the Trans-Blot Turbo (trademark) Transfer System (manufactured by BIO-RAD Laboratories). Next, the membrane was then blocked for 1 hour at room temperature with a blocking buffer (5% nonfat milk in Tris-buffered saline containing 0.05% Tween-20 (TBST)), and incubated overnight at 4° C. with a primary antibody against either an anti-human IL-1Ra antibody (1:100, mouse monoclonal antibody, manufactured by Santa Cruz Biotech) or anti-GAPDH (1:5000, mouse monoclonal antibody, manufactured by Sigma-Aldrich). After washing the membranes three times with TBST (10 min per wash), the membrane was incubated with an HRP-conjugated goat anti-mouse secondary antibody (Promega). The membranes were subsequently incubated in the SuperSignal (trademark) mWest Dura Extended Duration Substrate (Thermo Fisher Scientific) to enhance chemiluminescence. Finally, the blot was exposed for 2 minutes and detected using the iBright (trademark) CL1500 Imaging System (Thermo Fisher Scientific).

As shown in FIG. 2B, the IL-1Ra protein expression was confirmed 24 hours after administration of the IL-1Ra mRNA.

FIGS. 2A and 2B show that the IL-1Ra mRNA was delivered to the TMJ cartilage, where it produced protein. It was also shown that the protein can be expressed from the delivered IL-1Ra mRNA for more than 1 day. In other words, when a PIC micelle solution containing the IL-1Ra mRNA is administered, the mRNA and protein expressions are sustained for a longer period of time compared to anakinra (a recombinant protein formulation), which has a half-life of only 4 hours.

### Example 2-4: Evaluation of Joint Pain after Administration of the IL-1Ra mRNA

Joint pain caused by administration of a PIC micelle solution containing the IL-1Ra mRNA to TMJOA-induced rats was evaluated.

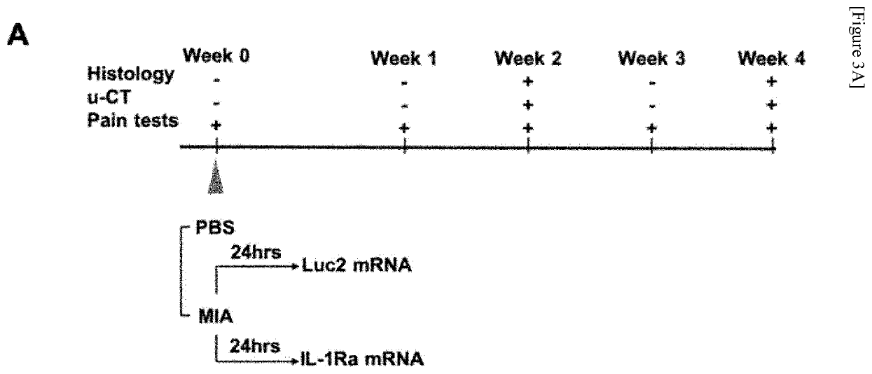

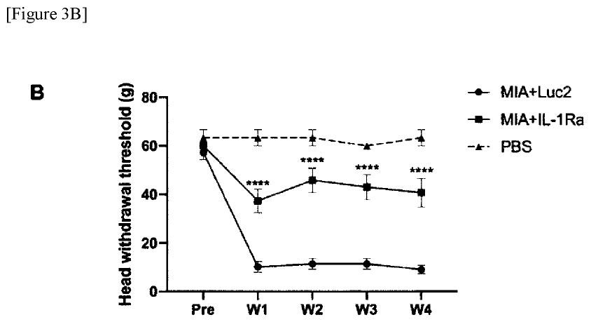

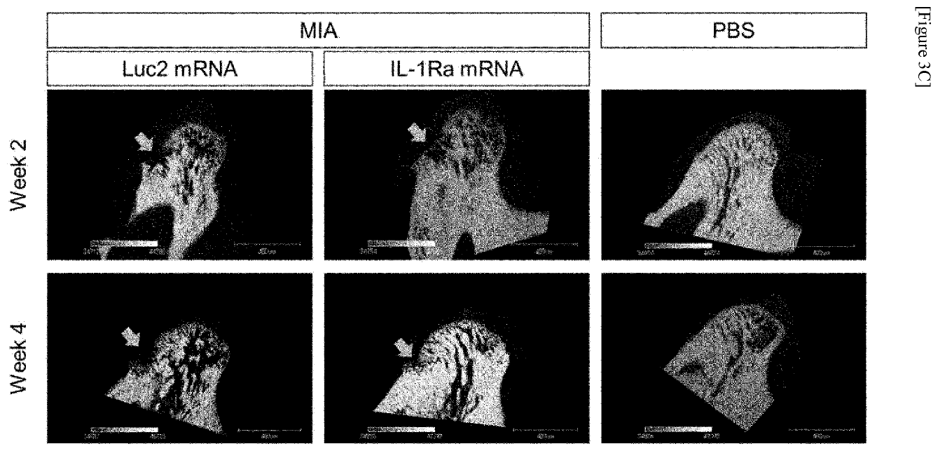

FIG. 3A shows a schematic diagram illustrating the process for evaluation. 0.5 mg of MIA dissolved in 50 μL of PBS was injected into the superior joint cavity of the temporomandibular joint (TMJ) of rats to induce temporomandibular joint osteoarthritis (TMJOA). Twenty-four hours later, 2.5 μg of the IL-1Ra mRNA was administered into the temporomandibular joint of the rat in which TMJOA had been induced (also described as “MIA+IL-1Ra”). As a control, 50 μL of a PIC micelle solution containing 2.5 μg of luciferase 2 (Luc2) mRNA was injected into the temporomandibular joint of rats in which TMJOA had been induced (also described as “MIA+Luc2”). In addition, as a normal TMJ model, no MIA but only PBS was injected into the superior joint cavity of the rat TMJ (also referred to as “PBS”). Assessment of joint pain (Pain tests) was performed weekly. TMJ samples were also collected 2 weeks and 4 weeks after mRNA administration for histological analysis (Histology) and micro-CT (μ-CT).

FIG. 3B shows the results of pain behavioral testing as assessed by head withdrawal threshold (HWT). The horizontal axis in FIG. 3B represents the time elapsed since injection of the PIC micelle solution containing mRNA or PBS only, and W1, W2, W3, and W4 represent the time after 1 week, 2 weeks, 3 weeks, and 4 weeks, respectively. The vertical axis represents the calculated head withdrawal threshold (HWT), indicating that higher the value, less the pain.

### (Measurement of Head Withdrawal Threshold (HWT))

The following procedure was used to measure the head withdrawal threshold (HWT). von Frey microfilaments (Muromachi Machinery Co., Ltd.) were used to test the threshold for the escape motion in response to mechanical stimulation of the temporomandibular joint from the minimum force of the filament, and the head withdrawal threshold (HWT) was measured. Tests were performed at least three times for each rat. HWT was calculated as the mean of 6 rats per group (total of 12 joints/group). Data are expressed as mean±SEM (standard error of the mean). **** represents P<0.0001. In comparisons between the MIA+IL-1Ra group and the MIA+Luc2 group, two-way analysis of variance was performed followed by Tukey's multiple comparison test.

As shown in FIG. 3B, pain was greatly reduced in MIA+IL-1Ra rats compared to MIA+Luc2 rats from the first week after the IL-1Ra mRNA administration, and this effect was sustained through the fourth week.

### Example 2-5: Suppression of Degeneration and Destruction of the Subchondral Bone by IL-1Ra mRNA

FIG. 3C is an X-ray micro-CT image of the sagittal view of the condylar subchondral bone in rats administered with the Luc2 mRNA (negative control) or IL-1Ra mRNA, or in rats administered with PBS only (normal), 2 weeks and 4 weeks after administration of each to the temporomandibular joint of rats in which TMJOA had been induced by administering MIA. Arrows in the image indicate bone erosion on the condylar surface

Evaluation by micro-CT (micro-computed tomography) was performed using the procedure below. Two and four weeks after injection of the PIC micelle solution containing mRNA, rats were euthanized and bilateral TMJs were dissected. The excised TMJ condylar heads were fixed overnight in 4% paraformaldehyde and analyzed using a high-resolution micro-computed tomography (micro-CT) system (inspeXio SMX-100CT, Shimadzu Corporation). The samples were scanned at 90 kV and 65 μA with an effective pixel size of 8 μm. Sagittal images of the condylar heads were reconstructed.

As shown in FIG. 3C, bone erosion was observed on the condylar surface of the subchondral bone of the TMJ to which MIA was administered, but the size of the bone destruction was smaller in the TMJ to which the IL-1Ra mRNA was administered compared with the TMJ to which the Luc2 mRNA was administered. Thus, the results indicated that the IL-1Ra mRNA administration suppresses degeneration and destruction of the subchondral bone.

### Example 2-6: Suppression of the Articular Cartilage Degeneration by IL-1Ra mRNA Administration

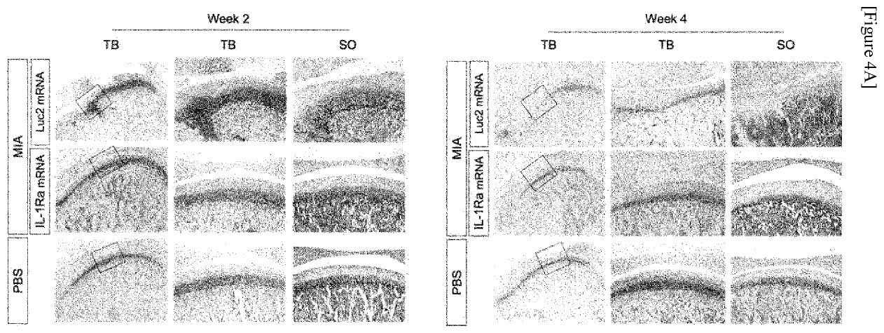

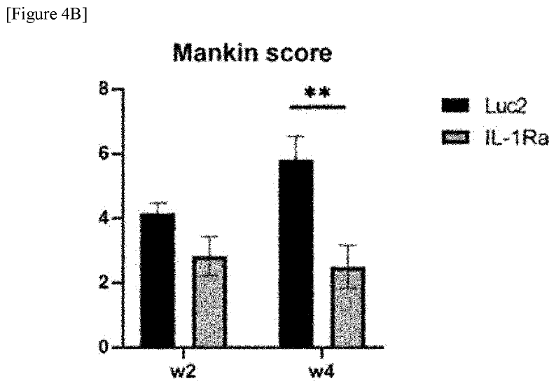

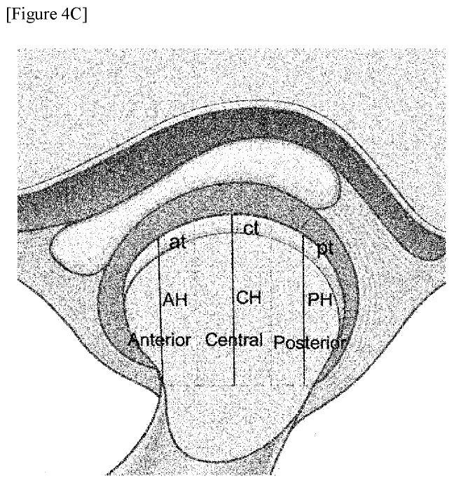

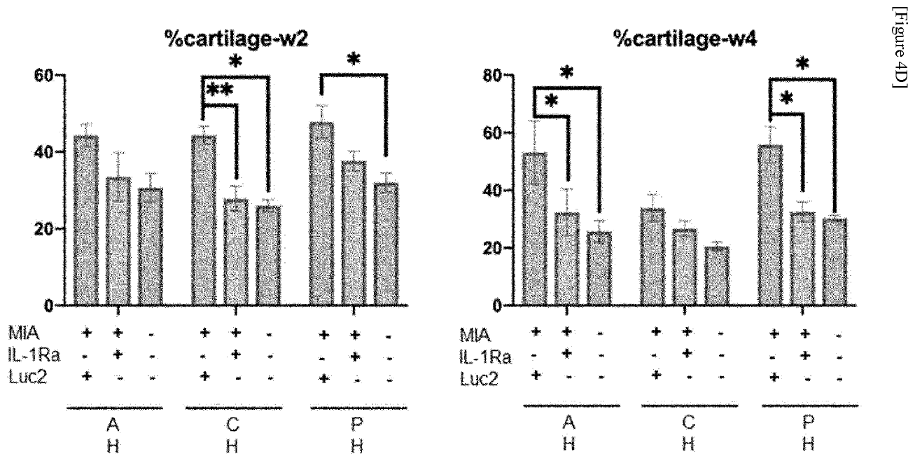

FIGS. 4A through 4D show the results of histological analysis of cartilage after administration of a PIC micelle solution containing the IL-1Ra mRNA to rats in which TMJOA had been induced. In these experiments, TMJ samples that were obtained 2 weeks and 4 weeks after injection of 2.5 μg of the IL-1Ra mRNA or luciferase 2 (Luc2) mRNA into the temporomandibular joint of rats as described above in (Example 2-5) were used. TMJ samples administered with PBS only were used as samples of normal cartilage. FIG. 4A shows images of sections stained with hematoxylin and eosin (HE), toluidine blue (TB), and safranin-O (SO) (the number of joints is 6/group).

Staining with HE, TB, and SO was performed according to the procedure below. After micro-CT examination, the fixed TMJ samples were dehydrated in a 20% sucrose solution and embedded in carboxymethylcellulose for cryosectioning using the Kawamoto film method. For cartilage evaluation according to standard protocols, serial sections were cut sagittally at 3 μm and stained with toluidine blue (TB) and safranin-O (SO). Cartilage deterioration and repair were blindly evaluated on TB and SO stained sections by three independent observers using the Mankin scoring system. The TB-stained sections were also used to measure cartilage thickness in each region (described below) using the ImageJ software Version 1.53 (National Institutes of Health).

Two weeks after mRNA administration, the Luc2 mRNA administration group showed chondrocyte loss in the anterior region of the condyle, destruction of the osteochondral junction in the central region, and chondrocyte proliferation with dark matrix staining in the central and posterior regions (FIG. 4A). This is consistent with previous reports using the MIA-induced TMJOA model (see Wang, X. D., et al. (2012) Progression of cartilage degradation, bone resorption and pain in rat temporomandibular joint osteoarthritis induced by injection of iodoacetate. PLOS One 7, e45036. 10.1371/journal.pone.0045036.). On the other hand, the IL-1Ra mRNA administration group showed smaller changes in the cartilage and osteochondral junction than the Luc2 mRNA administration group, and regularly arranged layers of chondrocytes were observed with less chondrocyte proliferation in the central region (FIG. 4A).

FIG. 4B shows the Mankin score 2 weeks (W2) or 4 weeks (W4) after administering the IL-1Ra mRNA or Luc2 mRNA to rats in which TMJOA had been induced by MIA (the number of joints is 6/group). The Mankin score is an evaluation method in which normal cartilage is given a score of 0; and chondrocytes, cartilage surface, and staining are each scored to determine the degree of histological degeneration of the cartilage tissue, with higher values indicating greater degeneration (Mankin, H. J., et al. (1971). Biochemical and metabolic abnormalities in articular cartilage from osteo-arthritic human hips. II. Correlation of morphology with biochemical and metabolic data. J Bone Joint Surg Am 53, 523-537.). As shown in FIG. 4B, although there was no significant difference at 2 weeks after mRNA administration, the IL-1Ra mRNA administration group showed lower scores than the Luc2 mRNA administration group, and the scores were even lower by a significant difference at 4 weeks (FIG. 4B). This indicates that the degeneration of articular cartilage is continuously suppressed by administration of the IL-1Ra mRNA.

FIG. 4C shows a schematic diagram for measuring the cartilage thickness of the condylar heads of the rat's temporomandibular joint. AH represents the height of the anterior region of the condylar head, CH represents the height of the center of the condylar head, and PH represents the height of the posterior region of the condylar head. at represents the thickness of the cartilage in the anterior region, ct represents the thickness of the cartilage in the center, and pt represents the thickness of the cartilage in the posterior region. In each of these regions, the thickness of the cartilage is shown as a percentage of the height of the condyle head (% at, % ct, % pt, see FIG. 4D). These percentages of cartilage thickness represent the thickening of the cartilage surface as a result of fibrosis.

FIG. 4D shows the thickness of the cartilage in the temporomandibular joint at 2 weeks (W2) and 4 weeks (W4) after mRNA administration. Cartilage thickness was measured in each of the regions shown in FIG. 4C as described above (the number of joints is 6/group). Data are expressed as mean±SEM (standard error of the mean). * denotes P<0.05 and ** denotes P<0.01. Two-way analysis of variance was performed followed by Tukey's multiple comparison test for analysis. The percentage of cartilage in the TMJ of rats in the MIA+IL-1Ra group was at the same level as the cartilage in the normal TMJ (without MIA administration), whereas the percentage of cartilage was greater in the MIA+Luc2 group. Thus, the MIA+Luc2 group showed greater thickening due to surface fibrosis and greater cartilage degeneration, whereas cartilage degeneration was suppressed in the MIA+IL-1Ra group.

### Example 2-7: Suppression of Inflammation Induced by Osteoarthritis (OA) as a Result of IL-1Ra mRNA Administration

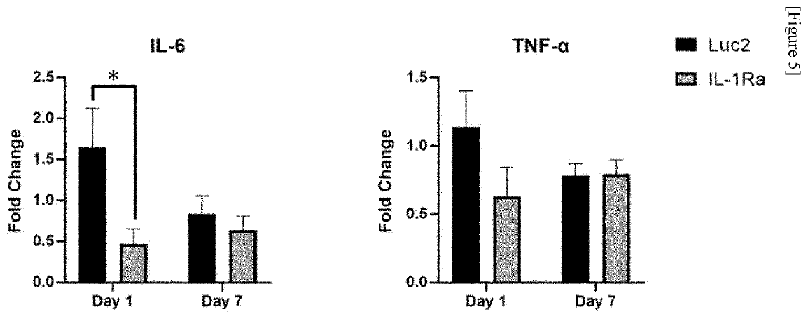

To investigate the regulatory mechanism of immune response in treatment using the IL-1Ra mRNA, the gene expression of inflammatory cytokines in cells of MIA+Luc2 mRNA, MIA+IL-1Ra mRNA, and normal TMJ cartilage was evaluated by qRT-PCR. FIG. 5 shows the expression levels of interleukin (IL)-6 and tumor necrosis factor (TNF)-α 1 and 7 days after mRNA administration (primer set is SEQ ID NOs: 6 to 9). IL-6 and TNF-α are proinflammatory cytokines. In FIG. 5, the vertical axis represents the expression level of each gene relative to its expression level in normal joints. The number of animals per group is 6, and * represents P<0.05. Two-way analysis of variance was performed followed by Tukey's multiple comparison test. The expression levels were normalized by the expression level of β-actin (primer set is SEQ ID NOs.: 4 and 5).

As shown in FIG. 5, the expression level of IL-6 in the MIA+Luc2 mRNA administration group was approximately 1.5 times that in normal joints one day after the mRNA administration. In contrast, the IL-6 expression level in the MIA+IL-1Ra mRNA administration group was significantly lower than in the Luc2 mRNA administration group. On the other hand, the IL-6 expression decreased in both groups 7 days after the mRNA administration.

The expression level of TNF-α was lower in the IL-1Ra mRNA administration group than in the Luc2 mRNA administration group 1 day after the mRNA administration, although the difference was not significant. These results suggest that the IL-1Ra mRNA suppresses cartilage inflammation by inhibiting cytokines downstream of IL-1.

### <Example 3> Administration of the IL-1Ra mRNA and Runx1 mRNA to an Animal Model of Knee OA (Rat)

### Example 3-1: Preparation of a PIC Solution Encapsulating mRNA

As the cationic polymer, a PEG-PAsp (DET) block copolymer produced in the same manner as described above (Example 1-1) was used. For mRNA, the IL-1Ra mRNA and Runx1 mRNA prepared above (Example 1-2) were used. The PEG-PAsp (DET) block copolymer and each mRNA were dissolved in 10 mM HEPES buffer (pH 7.3). A solution of the PEG-PAsp (DET) block copolymer was mixed with the mRNA solution, so that the mixing ratio (N/P) of the amino group of the cationic group in the block copolymer (N) to the phosphate group in the mRNA (P) was 3 and the final concentration of mRNA was 200 μg/mL. Three types of mRNA were prepared: IL-1Ra mRNA only, Runx1 mRNA only, and a mixture of IL-1Ra mRNA and Runx1 mRNA (mixing weight ratio: 1:1); and a PIC micelle solution was prepared for each.

### Example 3-2: Preparation of Rat Knee OA Model and mRNA Administration

OA was induced in the right knee joint of 8-week-old Slc:SD rats (Sankyo Lab Service Co., Ltd.) by intra-articular injection of 50 μL of a saline solution containing 0.25 mg of monoiodoacetic acid (MIA) under general anesthesia. On day 1, 4, 7 and 11 following MIA administration, the IL-1Ra mRNA or Runx1 mRNA (each alone or both) was administered intra-articularly to the right knee joint using a PIC micelle solution prepared in (Example 3-1). The mRNA was administered by intra-articularly injecting 50 μL of a PIC micelle solution containing 10 μg of mRNA. When administering a mixture of the IL-1Ra mRNA and Runx1 mRNA, a PIC micelle solution containing 5 μg of each mRNA (the total amount of mRNA was the same) was used.

### Example 3-3: Evaluation of Knee Joint Pain (Incapacitance Test)

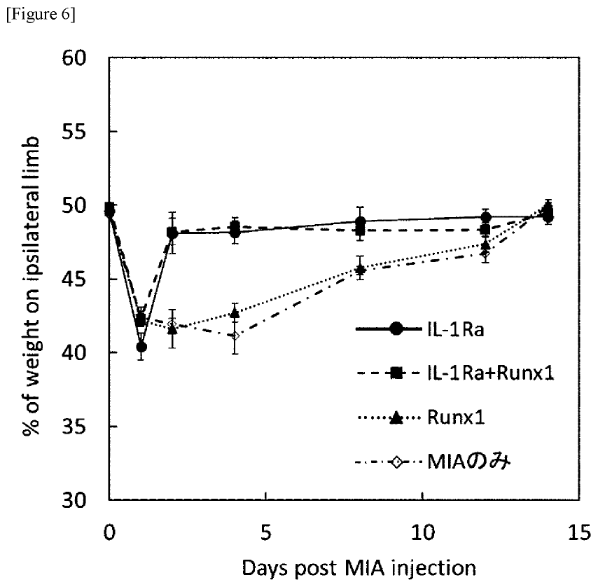

OA knee pain was evaluated using an incapacitance test (BIO-SWB-TOUCH, Bio Research Center Inc.), which measures the load on the rat's lower limb separately on the left and right sides. Measurements were taken before (day 0), and on day 1, day 2, day 4, day 7, day 11, and day 14 after MIA administration, or before mRNA administration if the day coincided with the mRNA administration. Measurements were taken three times per animal for 10 seconds continuously, and the average value was calculated. Using the values for the right and left lower limbs, the ratio of the weight on the affected lower limb to the total weight including the healthy side (% of Weight on Ipsilateral Limb) was calculated (Equation (1) below), and the changes over time were plotted (FIG. 6). Statistical processing was performed by one-way analysis of variance followed by Tukey's test to evaluate each group. Differences were considered significant at the p <0.05 level.

$$\begin{matrix}[ Math 1 ] & \\ \% of Weight in Ipsilateral Limb = \frac{right}{Right + Left} \times 100 & Equation ( 1 )\end{matrix}$$

As mentioned above, the incapacitance test is a method that measures separately the weight on both lower limbs and evaluates knee joint pain based on the difference between the left and right sides. Under the normal condition, the value calculated by the above equation (1) is 50%, and the greater the pain, the smaller this value becomes. In the group administered with only the IL-1Ra mRNA or the group administered with mixed mRNA of the IL-1Ra mRNA and Runx1 mRNA, a pain-reducing effect was observed immediately after administration. On the other hand, the group administered with only Runx1 showed almost no difference from the group that received no mRNA (MIA administration only), indicating that Runx1 alone was not effective in alleviating pain.

### Example 3-4: Histological Evaluation

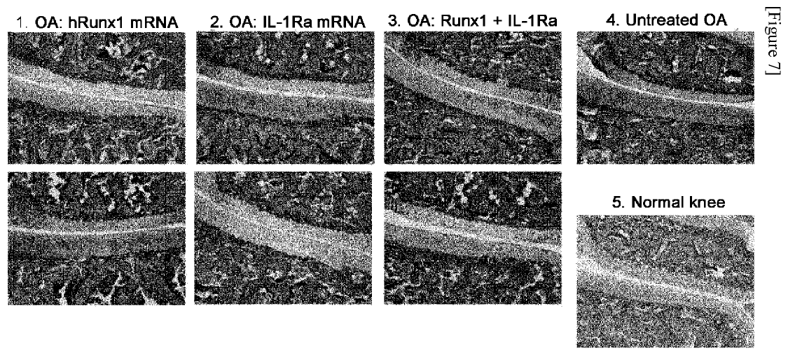

After induction of knee OA and mRNA administration as described above (Example 3-2), histological evaluation was performed 2 weeks after MIA administration. Mice were euthanized, and the right knee joint was excised in one piece and frozen with hexane dry ice. Next, the frozen specimen was placed in an embedding medium (SCEM, manufactured by SECTION-LAB) and frozen with hexane dry ice. This frozen specimen was trimmed using a tungsten carbide blade in a cryomicrotome (CM1950 Leica Biosystems) kept at −25° C. After trimming, an adhesive film was attached to the sample to prepare frozen sections with a thickness of 4 μm. The thawed frozen sections were immersed in 100% ethanol for 10 seconds and then in distilled water for 1 minute. Fast Green Safranin O staining was performed with Carrazzi's Hematoxylin solution for 1 minute, followed by washing in running water for 1 minute. After that, staining was carried out in the following order: 10 minutes of staining with 0.08% Fast Green aqueous solution, 10 seconds with 1% acetic acid, 15 minutes with 0.1% Safranin O solution, and 10 seconds with 1% acetic acid, followed by a final wash in distilled water for 10 seconds. After staining, mounting was performed using SCMM-G1 (manufactured by SECTION-LAB), a glycerin-based mounting medium. Photographs were taken using an all-in-one fluorescence microscope (BZ-X810, Keyence). The results are shown in FIG. 7.

In FIG. 7, it was observed that while the overall changes were mild, the findings of irregular cartilage surface layers and decreased staining were observed in the untreated group after OA induction, whereas the changes were relatively suppressed when only the Runx1 mRNA or Runx1 mRNA+IL-1Ra mRNA was administered. Therefore, the effect of suppressing cartilage degeneration is more strongly achieved with the Runx1 mRNA, suggesting that it is effective to administer the IL-1Ra mRNA and Runx1 mRNA in combination for the treatment that reduces pain caused by OA and suppresses cartilage degeneration.

In the above example, the primer sequences used for qRT-PCR are shown in Table 1.

TABLE 1

| | | |
|---|---|---|
| Gene | Forward | Reverse |

Human

| | | |
|---|---|---|
| IL-1Rα | 5′-AACCTTCTACCTGCGGAACA-3′ | 5′-GCCAGACTTCACACAGCTCA-3′ |
| | (SEQ ID NO: 2) | (SEQ ID NO: 3) |

Rat

| | | |
|---|---|---|
| β-Actin | 5′-CACCCGCGAGTACAACCTTCT-3′ | 5′-TCGTCATCCATGGCGAACTGG-3′ |
| | (SEQ ID NO: 4) | (SEQ ID NO: 5) |
| IL-6 | 5′-GTTTCTCTCCGCAAGAGACTTC-3′ | 5′-TGTGGGTGGTATCCTCTGTGA-3′ |
| | (SEQ ID NO: 6) | (SEQ ID NO: 7) |
| TNF-α | 5′-ACTGAACTTCGGGGTGATCG-3′ | 5′-TCCGCTTGGTGGTTTGCTAC-3′ |
| | (SEQ ID NO: 8) | (SEQ ID NO: 9) |

### <Example 4> Evaluation of OA Knee Pain

The observation period described above in FIG. 6 was extended to 4 weeks. That is, days 21 and 28 were added. Control (healthy rats without OA induction) data was added as a sample (N=4). The method and such are the same as in FIG. 6. Statistical processing is at p<0.05*, 0.01**, 0.001***, and 0.0001****.

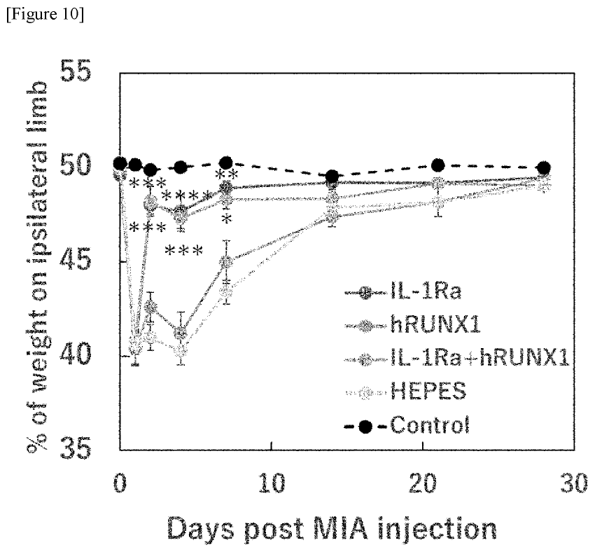

The results are shown in FIG. 10. The same results as in FIG. 6 were observed.

### <Example 5> Evaluation of the Degree of Swelling in OA Knees (Knee Diameter)

The anteroposterior and lateral diameters of the knee joint were measured at the center of the patellar ligament, and the mean value was plotted as the diameter (N=4). Statistical processing is the same as in Example 4.

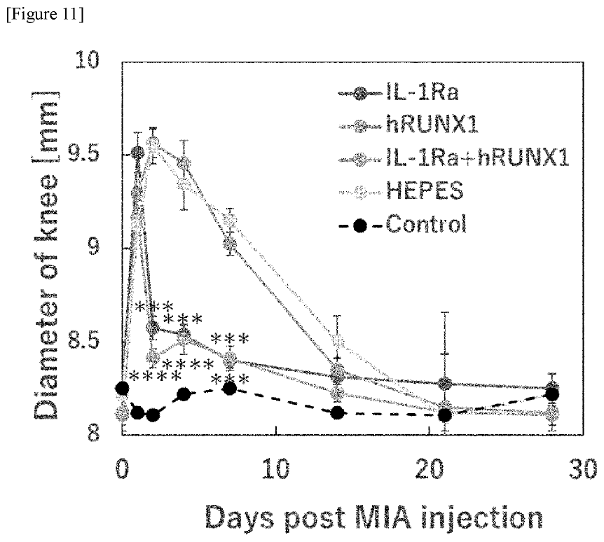

The results are shown in FIG. 11. As shown in FIG. 11, a significant effect of knee swelling reduction was observed in the IL-1Ra mRNA administration group (alone or in combination with the Runx1 mRNA).

### <Example 6> Histological Evaluation of OA Knees (4 Weeks after OA Induction)

The same experiment was performed by the same method as described above in FIG. 7 (evaluation after 2 weeks).

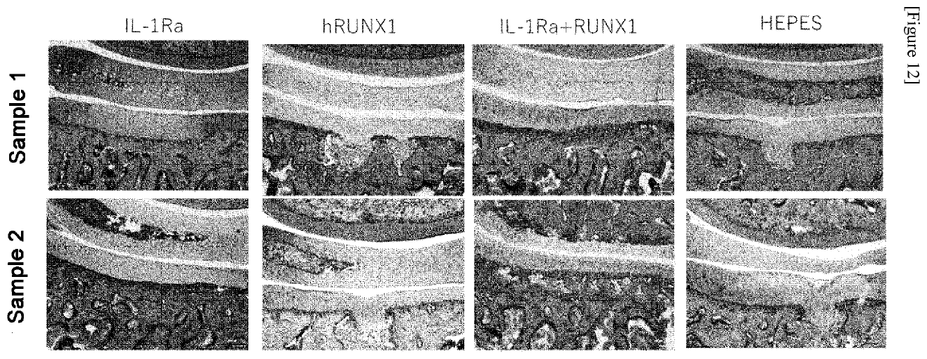

The results are shown in FIG. 12. As shown in FIG. 12, the properties of the cartilage surface layer and safranin O staining are well maintained in the Runx1 mRNA administration group (in particular, the group administered simultaneously with IL-1Ra). On the other hand, in the group not administered with the IL-1Ra mRNA, subchondral bone destruction (intra-articular inflammation→osteoclast induction) was clearly observed, and even in the group administered with the Runx1 mRNA alone, bone degeneration caused irregularities in the cartilage surface layer. Note that Runx1 is a cartilage-specific transcription factor and does not directly contribute to bone healing. This suggests that in conditions where bone destruction occurs and major changes in morphology occur, the Runx1 mRNA alone is not sufficient to suppress the progression of cartilage degeneration. Therefore, when looking at the joint tissue as a whole, controlling inflammation (IL-1Ra) is important to prevent bone destruction, and it is further considered effective to administer the Runx1 mRNA in combination to for the purpose of suppressing cartilage degeneration.

The present application claims priority based on Japanese Application JP-A2022-122392 filed on Jul. 30, 2022, and also claims priority based on Japanese Application JP-A2022-131285 filed on Aug. 19, 2022; and the disclosures are incorporated herein by its entirety.

Although the present invention has been described above with reference to the embodiments and examples, the present invention is not limited to the above embodiments and examples. The configuration and details of the present invention can be modified in various ways that can be understood by those skilled in the art within the scope of the present invention.

1. A pharmaceutical composition for use in the treatment and/or prevention of a joint disease, the pharmaceutical composition comprising a polyion complex comprising: (a) a cationic polymer comprising a cationic unnatural amino acid as a monomer unit, wherein the cationic unnatural amino acid is an amino acid having a group represented by —(NH—(CH₂)₂)ₚ—NH₂ (wherein p is 2, 3 or 4) as a side chain, and (b) an mRNA encoding IL-1Ra.

2. The pharmaceutical composition according to claim 1, further comprising an mRNA encoding Runx1.

3. The pharmaceutical composition according to claim 1, wherein the cationic polymer is a block copolymer comprising a block based on polyethylene glycol and a block containing the cationic unnatural amino acid as a monomer unit.

4. The pharmaceutical composition according to claim 1, wherein the cationic polymer is represented by formula (I) below: 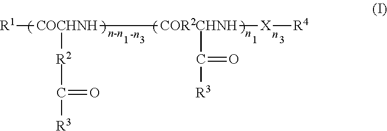
wherein in formula (I),
R¹ is a group based on polyethylene glycol, and the polyethylene glycol and the adjacent amino acid may be bonded via a linker;
R² is a methylene group or an ethylene group;
R³ is a group represented by —(NH—(CH₂)₂)ₚ—NH₂, and p is 2, 3 or 4;
R⁴ is hydrogen, a protecting group, a hydrophobic group or a polymerizable group;
X is a group based on a cationic amino acid;
n is any integer of 2 to 5000;
n₁ is any integer of 0 to 5000;
n₃ is any integer of 0 to 5000; and
n-n₁-n₃ is any integer greater than or equal to 0; and
wherein each repeating unit in the formula is shown in a specific order for convenience of description, but each repeating unit can exist in any order, and each repeating unit may exist randomly, and each repeating unit may be the same or different.

5. The pharmaceutical composition according to claim 1, wherein the joint disease is arthritis, osteoarthritis, or rheumatoid arthritis.

6. A method for treating and/or preventing a joint disease in a subject in need thereof, the method comprising administering to the subject a polyion complex comprising: (a) a cationic polymer comprising a cationic unnatural amino acid as a monomer unit, wherein the cationic unnatural amino acid is an amino acid having a group represented by —(NH—(CH₂)₂)ₚ—NH₂ (wherein p is 2, 3 or 4) as a side chain, and (b) an mRNA encoding IL-1Ra.

7. The method according to claim 6, wherein the polyion complex further comprises an mRNA encoding Runx1.

8. The method according to claim 6, wherein the cationic polymer is a block copolymer comprising a block based on polyethylene glycol and a block containing the cationic unnatural amino acid as a monomer unit.

9. The method according to claim 6, wherein the cationic polymer is represented by formula (I) below: 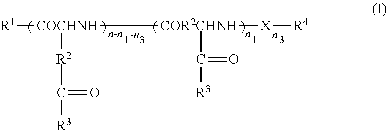
wherein in formula (I),
R¹ is a group based on polyethylene glycol, and the polyethylene glycol and the adjacent amino acid may be bonded via a linker;
R² is a methylene group or an ethylene group;
R³ is a group represented by —(NH—(CH₂)₂)ₚ—NH₂, and p is 2, 3 or 4;
R⁴ is hydrogen, a protecting group, a hydrophobic group or a polymerizable group;
X is a group based on a cationic amino acid;
n is any integer of 2 to 5000;
n₁ is any integer of 0 to 5000;
n₃ is any integer of 0 to 5000; and
n-n₁-n₃ is any integer greater than or equal to 0; and
wherein each repeating unit in the formula is shown in a specific order for convenience of description, but each repeating unit can exist in any order, and each repeating unit may exist randomly, and each repeating unit may be the same or different.

10. The method according to claim 6, wherein the joint disease is arthritis, osteoarthritis, or rheumatoid arthritis.


SEQUENCE LISTING (10 sequences)

SEQ ID NO: 1 - DNA, 586 bp, Homo sapiens
  source 1..586: mol_type = genomic DNA
  misc_feature 17..25
  gene 23..565
gcaggtcgactctagagccaccatggctctggccgacctgtatgaagaaggtggcggcggaggcggagagggcgaagataatgccgacagcaaagagacaatctgcagacccagcggccggaagtcctctaagatgcaggccttcagaatctgggacgtgaaccagaaaaccttctacctgcggaacaatcagctggtggccggctatctgcagggccccaatgtgaacctggaagagaagatcgacgtggtgcccatcgagccccacgctctgtttcttggaatccacggcggcaagatgtgcctgagctgtgtgaagtctggcgacgagacacggctgcagctggaagccgtgaacatcaccgacctgagcgagaaccggaagcaggacaagagattcgccttcatcagaagcgacagcggccccaccacaagctttgagtctgctgcttgccctggctggttcctgtgtacagccatggaagccgaccagcctgtgtctctgaccaacatgcctgacgagggcgtgatggtcaccaagttctacttccaagaggacgagtagggtaccgagctcgaattcaaa

SEQ ID NO: 2 - DNA, 20 bp, synthetic construct
  misc_feature 1..20: note = Primer sequence
  source 1..20: mol_type = other DNA
aaccttctacctgcggaaca

SEQ ID NO: 3 - DNA, 20 bp, synthetic construct
  misc_feature 1..20: note = Primer sequence
  source 1..20: mol_type = other DNA
gccagacttcacacagctca

SEQ ID NO: 4 - DNA, 21 bp, synthetic construct
  misc_feature 1..21: note = Primer sequence
  source 1..21: mol_type = other DNA
cacccgcgagtacaaccttct

SEQ ID NO: 5 - DNA, 21 bp, synthetic construct
  misc_feature 1..21: note = Primer sequence
  source 1..21: mol_type = other DNA
tcgtcatccatggcgaactgg

SEQ ID NO: 6 - DNA, 22 bp, synthetic construct
  misc_feature 1..22: note = Primer sequence
  source 1..22: mol_type = other DNA
gtttctctccgcaagagacttc

SEQ ID NO: 7 - DNA, 21 bp, synthetic construct
  misc_feature 1..21: note = Primer sequence
  source 1..21: mol_type = other DNA
tgtgggtggtatcctctgtga

SEQ ID NO: 8 - DNA, 20 bp, synthetic construct
  misc_feature 1..20: note = Primer sequence
  source 1..20: mol_type = other DNA
actgaacttcggggtgatcg

SEQ ID NO: 9 - DNA, 20 bp, synthetic construct
  misc_feature 1..20: note = Primer sequence
  source 1..20: mol_type = other DNA
tccgcttggtggtttgctac

SEQ ID NO: 10 - DNA, 1740 bp, Homo sapiens
  source 1..1740: mol_type = genomic DNA
  primer_bind 3..30
  primer_bind 11..30
  misc_feature 79..84
  gene 85..1527
cagtgaattgtaatacgactcactataaggcgaattaagagagaaaagaagagtaagaagaaatataagacaccggtcgccaccatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaatctagaccttctgcggggcttgccttctggccatgcccttcttctctcccttgcacctgtacctcttggtctttgaataaagcctgagtaggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa